(19) United States
(12) Patent Application Publication
(10) Pub. No.: US 2012/0207715 A1
(43) Pub. Date: Aug. 16, 2012

(54) METHODS AND SYSTEMS FOR STORING AND PROLONGING VIABILITY OF MATRIX DEPENDENT CELLS

(75) Inventor: Raphael GORODETSKY, Jerusalem (IL)

(73) Assignee: Hadasit Medical Research Services & Development Ltd., Jerusalem (IL)

(21) Appl. No.: 13/368,408

(22) Filed: Feb. 8, 2012

Related U.S. Application Data
(60) Provisional application No. 61/441,307, filed on Feb. 10, 2011.

(51) Int. Cl. A61K 35/12; A61P 9/00; A61P 19/04; C12M 1/00; C12N 5/0775

(52) U.S. Cl. 424/93.7; 435/307.1; 435/374

(57) ABSTRACT

The present invention relates to systems, methods and storage media for preserving and prolonging viability of cultured matrix dependent cells including multipotent progenitor cells, such as mesenchymal stem cells. The system and method of the invention are effective in ambient room temperature and apply during storage and shipment of said cells. The storage medium of the invention comprises fibrin microbeads and culture medium, and is suitable for the maintenance and storage of matrix dependent cells. The methods of the invention comprise use of said system, for attaching matrix dependent cells to fibrin microbeads in culture so as to form cell-fibrin microbead complexes.

### REFERENCE TO CO-PENDING APPLICATIONS

Priority is claimed to U.S. Provisional Patent application No. 61/441,307, filed on Feb. 10, 2011.

### FIELD OF THE INVENTION

The present invention relates to systems, methods and storage media for preserving and prolonging viability of cultured matrix dependent cells including multipotent progenitor cells, such as mesenchymal stem cells. The storage medium of the invention comprises fibrin microbeads and culture medium, and is suitable for the maintenance and storage of matrix dependent cells. The methods of the invention comprise use of said system, for attaching matrix dependent cells to fibrin microbeads in culture so as to form cell-fibrin microbead complexes, and storing the cell-fibrin microbead complexes at ambient temperatures.

### BACKGROUND OF THE INVENTION

Cell-based treatment protocols frequently involve delays or prolonged time intervals between the preparation of a cell-based material, such as, a suspension or a matrix, which is intended for implantation into a subject, and the actual clinical procedure. It is well known that cells in suspension may not survive for extended time periods while being transferred in ambient conditions. To date, cell suspensions and artificially engineered tissues involve complicated and expensive set-up procedures for their maintenance, including special culture conditions requiring warming or cooling the culture to highly specific temperatures.

Fibrin microbeads (FMB) which are biodegradable protein based cell carriers that support expansion of matrix-dependent cells have been described by one of the inventors of the present invention, for example in U.S. Pat. Nos. 6,737,074; 6,503,731 and 6,150,505 and Gorodetsky et al., Methods Mol. Biol. 238, 11-24, 2004. Such FMB are further disclosed by one of the inventors of the present invention as being useful for culturing cells, including bone marrow-derived progenitor cells (e.g. Gorodetsky et al., J Invest Dermatol., 112: 866-872, 1999) and bone marrow-derived mesenchymal stem cells (e.g. Rivkin et al., Cloning Stem Cells 9, 157-175, 2007)

Other types of microbeads which may comprise fibrin include those with a relatively low degree of cross-linking (Senderoff et al., J Parenteral Sci Tech 1991, 45(1):2-6) and those prepared by cross-linking with glutaraldehyde (Ho et al., Drug Des. Deliv. 1990 December; 7(1):65-73)

Furthermore, biodegradable micro-spheres and micro-carriers based on poly(lactic-co-glycolic acid) have been proposed for preparing 3D cell cultures for eventual transplantation (see for example, Chung et al., Tissue Eng Part A 15, 1391-1400, 2009).

PCT publication Nos. WO 01/53324 and WO 2004/041298 of one of the inventors of the present invention disclose synthetic haptotactic peptides homologous to fragments of fibrinogen. According to these publications, attachment of said synthetic peptides to various matrices increases attachment of the matrices to matrix dependent cells.

PCT publication No. WO 2009/022340 of one of the inventors of the present invention discloses a pharmaceutical composition for sequestering cells in connective tissue comprising biocompatible, biodegradable scaffolding in the form of beads comprising hyaluronic acid and an amino acid sequence from human ficolin.

PCT publication No. WO 2004/076631 discloses a biologically active biomatrix composition derived from human amnions, which comprises laminin, collagen I and collagen IV, and may further comprise an extracellular matrix protein inter alia fibrin, and wherein the biomatrix may be coated on a microbead. According to the disclosure, the scaffold may further comprise an accessory cell such as a mesenchymal cell.

PCT publication No. WO 2003/083044 discloses a test system and method for using tissue analogs, the method comprising: (1) isolating the cells to be implanted from donor tissue; (2) seeding the cells onto a particulate microcarrier bead; (3) culturing the cells on the microcarriers to expand the number of cells; and (4) further culturing the cell-particle aggregates to form a tissue analog. According to the disclosure, the cells may be mesenchymal stem cells or pluripotent stem cells derived from bone marrow stroma, and the microcarrier beads may be prepared from fibrin. Further disclosed are kits for transporting frozen quantities of the tissue analog.

U.S. Patent Application Publication No. 2007/0116680 discloses methods for embedding stem cells within three-dimensional hydrogel microenvironments formed from naturally derived proteins, inter alia fibrinogen or fibrin. The disclosed methods involve suspending stem cells in solutions of matrix components, emulsifying the solutions in a hydrophobic phase, triggering gelation of the matrix components by changing the environmental conditions, and collection of the resulting hydrogel beads, which may be further cultured to promote directed differentiation of the embedded stem cells.

U.S. Patent Application Publication No. 2010/0279411 and U.S. Pat. No. 7,786,082 disclose a method for stimulating proliferation and promoting survival of mesenchymal or hematopoietic stem cells, or their progenitor cells before transplantation, the method comprising the steps of (i) contacting the cells with a first composition comprising placental alkaline phosphatase in a cell culture medium containing 0-10% serum, and (ii) harvesting said cells in a medium supplemented with a second composition comprising placental alkaline phosphatase.

### SUMMARY OF THE INVENTION

Cell preparations intended for cell therapy have to maintain cell viability between preparation in the laboratory and their clinical application in the treatment clinic or operation room. In addition, cells for research applications often involve cells transfer between research centers. Timing and means for cell conveyance between locations must be strategically planned, since exposure to varying environmental factors can compromise the clinical efficacy of cell-based treatments.

The system and method of the invention enable shipping cells at room temperature between laboratories and medical centers for prolonged time intervals, while maintaining their viability and compatibility, e.g. for research applications.

The present invention is based in part on the unexpected discovery that mesenchymal cells, including mesenchymal stem cells derived from bone marrow stroma, attached to fibrin microbeads (FMB), exhibit prolonged viability e.g. for at least 10 days, when stored at ambient temperatures. Moreover, the stored cells exhibited maximal survival rate and about 100% recovery. In contrast, cells grown on FMB in the same manner and then deep cooled under high pressure, or those maintained at 4° C., exhibited significantly poorer survival profiles.

It is to be understood that the term ‘ambient temperature’ as used herein includes temperatures within the range of about 18° C. to about 30° C. Thus, the methods of the invention do not require warming or cooling of the cells during storage. Advantageously, the invention enables transport of “ready-to-use” cells at room temperature, and thus has enormous practical implications for regenerative medicine, most significantly for streamlining the logistics associated with shipment of living cells for dispatch between laboratories or between preparative laboratory and clinical setting.

Without wishing to be bound by any particular theory or mechanism of action, the efficacy of the invention may be attributed to the ability of FMB to serve as a protective carrier for matrix-dependent cells, particularly under conditions of reduced oxygen tension. Such conditions may be accompanied by, but are not necessarily associated with, up-regulation of the expression of the gene for hypoxia induced factor 1α (HIF1α).

The teachings of the present invention are surprising over the commonly accepted dogma, which dictates that cell transfer and/or storage of living cells for extended periods requires their maintenance under well controlled conditions utilizing cooling systems or incubators, and in the case of 3D cultures usually under agitation. It is generally assumed that in order to maximize survival, stored cells, including storage during transportation between research centers, should be maintained at 4° C., as is practiced with donor organs for transplantation, or maintained warm at 37° C. in a dedicated incubator providing a regulated CO₂ atmosphere. Both of these approaches require a strategic temperature controlled setup.

In a first aspect, the invention provides a storage medium for preserving viability of isolated matrix dependent cells, the storage medium comprising fibrin microbeads and a culture medium.

In one embodiment, the fibrin microbeads are cross-linked fibrin microbeads comprising extensively cross-linked fibrin(ogen).

In another embodiment, the fibrin microbeads comprise at least one of a biodegradable polymer, an extracellular matrix component and a growth factor. Each possibility is a separate embodiment of the invention.

In yet another embodiment, the culture medium is a serum-containing culture medium or a serum-free culture medium. Each possibility is a separate embodiment of the invention.

In yet another embodiment, the fibrinogen is obtained from pooled plasma.

In yet another embodiment, the storage medium further comprises cells bound to the fibrin microbeads wherein the storage medium is stored in a receptacle. In yet another embodiment, the receptacle is substantially filled with the culture medium. In yet another embodiment, the volume of the receptacle that is unoccupied by the cells bound to fibrin microbeads is substantially filled with the culture medium.

In another aspect, the invention provides a method of preserving viability of isolated matrix dependent cells, the method comprising the steps of:
(i) providing a preparation of isolated matrix dependent cells;
(ii) culturing the cell preparation of (i) with fibrin microbeads in a culture medium under conditions permitting the cells to bind to the fibrin microbeads, thereby obtaining cell-fibrin microbead complexes; and
(iii) storing the cell-fibrin microbead complexes under sealed conditions at a temperature in the range of 16 to 32° C.

It is to be understood that while stored, according to the method of the invention, the cell-fibrin microbead complexes may be transferred from one location to another (e.g. from the laboratory to the clinic). Thus, storing as used herein refers to maintenance under the condition of the method of the invention, either during conveyance between different locations or while settled in one location.

In one embodiment, the storing in (iii) comprises storing the cells bound to fibrin microbeads under a culture medium in a suitable receptacle. In another embodiment, the storing in (iii) is carried out at a temperature in the range of 18 to 30° C. In yet another embodiment, the culture medium for storing the cells is different from the culture medium in (ii). In another embodiment, the receptacle is substantially filled with the culture medium. In yet another embodiment, the volume of the receptacle that is unoccupied by the cells bound to fibrin microbeads is substantially filled with the culture medium.

In yet another embodiment, the method further comprises separating the cells bound to the fibrin microbeads obtained in (ii) from unbound cells and unbound fibrin microbeads, prior to storing.

In yet another embodiment, the matrix dependent cells are selected from the group consisting of differentiated cells and multipotent progenitor cells having the capability of differentiating into several different cell types.

In yet another embodiment, the differentiated cells are selected from, but are not limited to, the group consisting of endothelial cells, epithelial cells, smooth muscle cells, skin fibroblasts, neuronal cells, cardiac cells, hepatic cells and pancreatic cells. Each possibility is a separate embodiment of the invention. In a particular embodiment, the differentiated cells are endothelial cells.

In yet another embodiment, the preparation of isolated matrix dependent cells comprises multipotent progenitor cells. In a particular embodiment, the preparation of isolated matrix dependent cells is enriched for multipotent progenitor cells.

In yet another embodiment, the multipotent progenitor cells are mesenchymal cells. In yet another embodiment, the mesenchymal cells are selected from the group consisting of mesenchymal stem cells, skin fibroblasts, myofibroblasts, smooth muscle cells, fibrocytes, endothelial cells, amnion mesenchymal cells, chorion mesenchymal cells and transgene-activated mesenchymal cells. Each possibility is a separate embodiment of the invention.

In yet another embodiment, the mesenchymal cells are human mesenchymal cells. In yet another embodiment, the mesenchymal cells are adult human mesenchymal cells.

In yet another embodiment, the mesenchymal cells are mesenchymal stem cells obtained from a human tissue or cell source selected from the group consisting of bone marrow stroma and umbilical cord blood. Each possibility is a separate embodiment of the invention.

In yet another embodiment, the mesenchymal stem cells are obtained from a cell type selected from the group consisting of bone marrow stroma cells and umbilical cord blood cells. Each possibility is a separate embodiment of the invention.

In yet another embodiment, the mesenchymal stem cells are capable of differentiating into a cell type selected from the group consisting of chondrocytes, osteoblasts, adipocytes and myocytes.

In yet another embodiment, the mesenchymal cells are human fibroblasts. In a particular embodiment, the human fibroblasts are selected from the group consisting of fibroblasts adult fibroblasts and foreskin fibroblasts. In another particular embodiment, the mesenchymal cells are mesenchymal stem cells isolated from human bone marrow stroma.

In yet another embodiment, the cells in (i) are cultured multipotent progenitor cells. In a particular embodiment, the cultured multipotent progenitor cells are those previously cultured in the presence of fibrin microbeads.

In yet another embodiment, the cell preparation in (i) is enriched for viable mesenchymal stem cells.

In yet another embodiment, the cell preparation enriched for viable mesenchymal cells is obtained by a process comprising (a) culturing cells from a tissue or cell source with fibrin microbeads in a culture medium under conditions permitting the cells to bind to and proliferate on the fibrin microbeads; and optionally, (b) separating the cells bound to the fibrin microbeads obtained in (a) so as to obtain a preparation of isolated viable mesenchymal stem cells.

In yet another embodiment, step (b) comprises culturing the mesenchymal stem cells obtained in (a) on a plastic surface so as to obtain a monolayer, and optionally passaging the cells.

In yet another embodiment, the conditions permitting the cells to bind to the fibrin microbeads comprise rotary or oscillating incubation at 35 to 37° C., in an environment containing about 21% oxygen and between about 5-10% CO₂. In yet another embodiment, the temperature in (iii) is room temperature. In yet another embodiment, the temperature in (iii) is in the range of 16 to 32° C. In yet another embodiment, the temperature in (iii) is in the range of 18 to 30° C. In yet another embodiment, the temperature in (iii) is in the range of 20 to 26° C. In yet another embodiment, the temperature in (iii) is in the range of 23 to 26° C.

In yet another embodiment, the storing is carried out at a ratio of cells bound to fibrin microbeads:culture medium in the range from 1:5 to 1:50 (v/v).

In yet another embodiment, the storing is carried out for a time period within the range of 3 days to 21 days.

It is to be understood that the storing in (iii) is carried out under normoxic conditions, wherein the receptacle is sealed after exposure to ambient oxygen conditions.

In yet another embodiment, preserving viability of the of isolated matrix dependent cells comprises arresting proliferation of the cells, thereby generally avoiding cell confluence which may result with a decrease in cell viability.

In yet another embodiment, preserving viability of the of isolated matrix dependent cells comprises maintaining the capability of multipotent progenitor cells to differentiate.

In yet another embodiment, the method further comprises (iv) incubating the cells obtained in step (iii) at 37° C. in a CO₂ incubator prior to use. In yet another embodiment, the incubating in step (iv) is carried out for up to 24 hours. In yet another embodiment, the incubating is carried out under about 21% oxygen and about 5-10% CO₂.

In yet another embodiment, the fibrin microbeads are cross-linked fibrin microbeads prepared in suspension in moderately heated oil in the absence of external crosslinkers.

In yet another embodiment, the fibrin microbeads comprise at least one of a biodegradable polymer, an extracellular matrix component and a growth factor. Each possibility is a separate embodiment of the invention.

In yet another embodiment, the culture medium further contains serum.

In yet another embodiment, the method further comprising implanting the stored cells in a subject in need thereof. In yet another embodiment, the cells for implantation are mesenchymal stem cells. In yet another embodiment, the cells are autologous, homologous (allogenic) or xenogenic in origin relative to the cells of said subject.

Preferably, prior to administering or implanting the cells, cells are allowed to recover from the storage medium, under appropriate conditions, for about 12 to 36 hours. Appropriate recovery conditions include incubation at 35-38° C. for 12 to 36 hours.

In yet another embodiment, said preparation of isolated matrix dependent cells comprises cells isolated from a tissue of said subject.

In yet another embodiment, the capacity, of the cells used for implantation, to differentiate is maintained

In yet another embodiment, the method further comprises separating the cells bound to the fibrin microbeads obtained in (ii) from unbound cells and unbound fibrin microbeads, prior to storing.

In yet another aspect, the invention further provides a preparation of isolated mesenchymal cells having extended viability, the preparation obtained by a process comprising the steps:
(i) providing cells from a tissue source;
(ii) culturing the cells of (i) with fibrin microbeads in a culture medium under conditions permitting mesenchymal cells to bind to the fibrin microbeads;
(iii) culturing the mesenchymal cells of (ii) with fibrin microbeads in a culture medium under conditions permitting the cells to bind to the fibrin microbeads; and
(iv) storing the cells bound to the fibrin microbeads obtained in (iii) under sealed conditions in a culture medium at a temperature in the range of 18 to 30° C.

In a particular embodiment, step (iv) further comprises culturing the mesenchymal cells bound to fibrin microbeads obtained in (iii) on a plastic surface so as to obtain a monolayer, and optionally passaging the cells.

In one embodiment, the method further comprises separating the cells bound to the fibrin microbeads obtained in (ii) from unbound cells and unbound fibrin microbeads, prior to storing.

In yet another aspect, the invention further provides a composition comprising a preparation of isolated multipotent progenitor cells having extended viability according to the invention, and a suitable cell carrier or medium. In particular embodiment, the composition further comprises at least one of a biodegradable polymer, an extracellular matrix component or a growth factor. Each possibility is a separate embodiment of the invention.

In particular embodiments, the invention provides a method for treating a cartilage or bone defect comprising administering a cell preparation of the invention, or a composition comprising a cell preparation of the invention, to a subject in need thereof. In particular embodiments, the methods comprise implanting an implantable device comprising the cell preparation of the invention, to a subject in need thereof. In particular embodiments, the cartilage or bone defect is associated with a condition selected from the group consisting of osteoarthritis and osteoporosis.

In other particular embodiments, the invention provides a method for treating a condition selected from spinal cord injury, periodontal disease and myocardial infarction, the method comprising administering a cell preparation of the invention, or a composition comprising a cell preparation of the invention to a subject in need thereof. Each possibility is a separate embodiment of the invention.

In particular embodiments, the cell preparation of the invention, or a composition comprising the cell preparation of the invention, or an implantable device comprising the cell preparation of the invention is for the treatment of a cartilage or bone defect. Each possibility is a separate embodiment of the invention.

In particular embodiments, a cell preparation of the invention, or a composition comprising a cell preparation of the invention, or an implantable device comprising a cell preparation of the invention is for the treatment of a condition selected from spinal cord injury, periodontal disease and myocardial infarction. Each possibility is a separate embodiment of the invention.

In particular embodiments, the cell preparation of the invention or a composition comprising a cell preparation of the invention, or an implantable device comprising a cell preparation of the invention is for the treatment of bone, cartilage or tissue reconstruction. Each possibility is a separate embodiment of the invention.

In particular embodiments, an implantable device comprising the cell preparation of the invention is for restoration, reconstruction, and/or replacement of tissues and/or organs.

In another aspect, the invention further provides a system for storage and conveyance of viable matrix dependent cells, ex vivo, the system comprising matrix dependent cell-fibrin microbead complexes, a container for holding said cell-fibrin microbead complexes in a liquid medium at a temperature in the range of 16 to 32° C., optionally, in the range of 18 to 30° C., culture medium and a liquid tight closure for sealing said container.

In one embodiment, the system further comprises serum. In another embodiment, the fibrin microbeads are cross-linked fibrin microbeads comprising extensively cross-linked fibrin(ogen). In yet another embodiment, the container is filled with the culture medium and the cell-fibrin microbead complexes, such that, the volume of the receptacle that is unoccupied by the cell-fibrin microbead complexes is filled with the culture medium.

Other objects, features and advantages of the present invention will become clear from the following description and drawings.

### BRIEF DESCRIPTION OF THE FIGURES

FIG. 1 shows the set-up of cell culture on FMB in cryotubes.

FIG. 2 shows survival of hMSC (human mesenchymal stem cells) loaded onto FMB following storage in cryotubes under sealed conditions for 6 days at different temperatures: RT (24° C.); cold (4° C.), and frozen (−20° C.).

FIGS. 3A and 3B shows cell survival of human mesenchymal stem cells (hMSC; A) and foreskin fibroblasts (FF; B) at different time points upon storage under different conditions.

FIG. 3C shows cell density of hMSC on FMB, as assessed by nuclei staining with propidium iodide. The lighter spots represent stained cell nuclei.

FIG. 4 shows survival of hMSC following loading onto FMB and maintenance under sealed conditions at RT in the presence of FCS-containing medium (white squares) or SFM (white circles); or maintenance in an incubator in the presence of FCS-containing medium (black squares) or SFM (black circles).

FIG. 5 shows HIF-1α expression in hMSC and FF, either grown onto fibrin microbeads (FMB; FIG. 5A) or grown in suspension in the absence of FMB (FIG. 5B), as assessed by real-time PCR, carried out at different time points up to 5 days and following a one day recovery period.

FIG. 6 shows total extractable RNA from the systems in FIG. 5. RNA was extracted from hMSC and FF following attachment of the cells to FMB and maintenance at RT (black circles and black squares, respectively); and from hMSC and FF grown in suspension in the absence of FMB (white circles and white squares, respectively).

FIG. 7 shows cell survival of bovine aortic endothelial cells (BAEC) at different time points upon storage under different conditions.

FIG. 8 shows survival of human MSC following growth onto FMB or polystyrene beads (Biosilon®) at 37° C. in a CO₂ incubator for 24 hours (attachment period), subsequent storage at RT for 6 days (storage period), followed by incubation at 37° C. for one day (recovery period).

### DETAILED DESCRIPTION OF THE INVENTION

The present invention provides a storage medium and a method for preserving cell viability in room temperature of different types of matrix dependent cells, including terminally differentiated cells and multipotent progenitor cells having the potential to differentiate into various cell lineages. The cells are stored while attached to fibrin microbeads (also referred to herein as “FMB”) following their isolation. FMB can also be used for initial isolation and expansion of populations of matrix dependent cells. The method of the invention enables multipotent progenitor cells, such as mesenchymal stem cells, to remain viable in storage for prolonged periods of time under minimally controlled conditions, e.g. at room temperature. The invention further provides methods of extending storage life and for promoting storage longevity of isolated mesenchymal cells. The methods of the invention enable ex vivo handling, transport and dispatch of mesenchymal cells, for example among different clinical facilities prior to their implantation into a subject. Based on the methods of the invention, the stored cells are maintained under sealed conditions in a liquid (culture) medium in a suitable receptacle at ambient environment, with no exogenous control of oxygen concentration.

The inventors of the present invention have surprisingly shown that mesenchymal cells and other matrix dependent cell types that are attached and grown on FMB, when sealed and stored at room temperature, exhibit a high rate of survival, as high as 100%, and maintain constant cell density, even when stored for long time intervals.

Thus, the invention provides a storage medium for extending storage life of isolated matrix dependent cells, comprising fibrin microbeads and a culture medium.

In one embodiment, the fibrin microbeads are cross-linked fibrin particles comprising extensively cross-linked fibrin(ogen). Each possibility is a separate embodiment of the invention.

In another embodiment, the fibrin microbeads comprise at least one of a biodegradable polymer, an extracellular matrix component and a growth factor. Each possibility is a separate embodiment of the invention.

Preferably, the fibrin microbeads do not contain any exogenous cross-linking agents such as glutaraldehyde that can damage certain biologically active sites that permit the microbeads to react with various types of cells.

Preferred fibrin microbeads contain extensive dehydrothermal cross-linking of fibrin(ogen) which renders the fibrin microbeads stable for prolonged periods in aqueous solution, a property which is particularly desirable for use as vehicles for culturing cells, and for other uses.

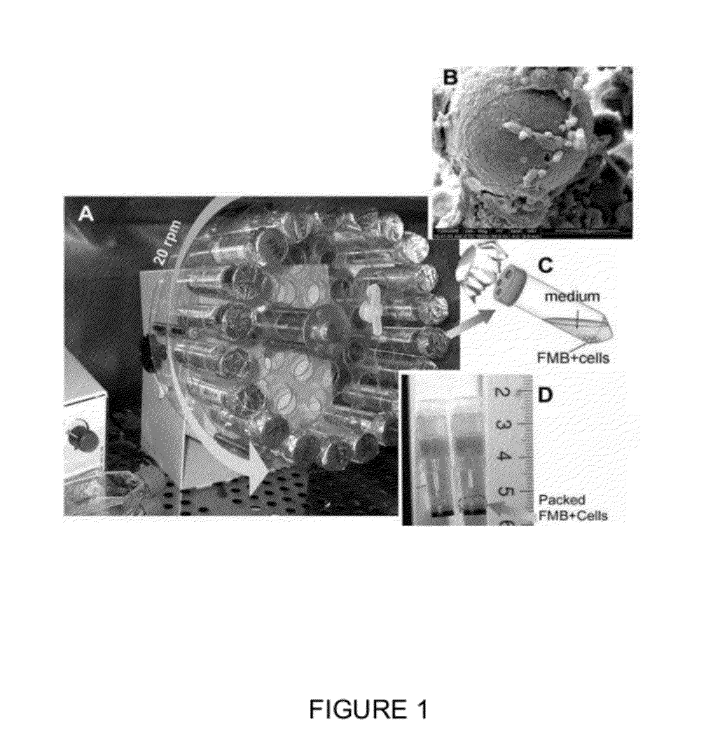

“Extensively cross-linked” means that the fibrin(ogen) contains at least 30% cross-linked fibrin(ogen), and more preferably at least 50% cross-linked fibrin(ogen). The extensive cross-linking of the fibrin microbeads of the present invention is believed to occur during their manufacture, which utilizes high temperatures that help denature the native fibrin(ogen) structure, specifically the D-domain, thereby exposing sites for cross-linking by factor XIII, which are not normally cross-linked by native conformers of fibrin(ogen) at ambient temperatures. The SDS-PAGE gel patterns (FIG. 1) show extensive cross-linking due to such factor XIII mediated reactions. The extensive cross-linking renders the microbeads of the present invention insoluble and stable in an aqueous environment, thus rendering the microbeads stable for cell culturing and other uses.

The fibrin microbeads according to the present invention may be produced in the following manner. First, an aqueous solution comprising fibrinogen, thrombin and factor XIII is prepared. This solution may be prepared by combining fibrinogen containing endogenous factor XIII with thrombin, by combining cryoprecipitate containing endogenous fibrinogen and endogenous factor XIII with thrombin, or by combining fibrinogen, factor XIII and thrombin individually into an aqueous solution. It also is within the confines of the present invention that equivalent proteases such as snake venom proteases (e.g. reptilase) may be used as an alternative to thrombin. The ratio of fibrinogen:thrombin:factor XIII in the aqueous solution is preferably 5-100 mg/mL:1-100 U/mL:1-50 U/mL, and most preferably 20-40 mg/mL:5-10 U/mL:2-20 U/mL. In addition to these proteins, the aqueous solution may also contain fibronectin and other blood-derived proteins that may be present in the fibrinogen and cryoprecipitate starting materials. If it is desired for the fibrin microbead to contain any bioactive agents, then those agents can be added into the fibrinogen or thrombin solutions prior to their mixing, or directly to the aqueous solution.

Next, prior to the onset of coagulation, the aqueous solution is introduced into an oil heated to a temperature in the range of about 50-80° C. to form an emulsion. A hydrophobic organic solvent such as isooctane also may be included in the oil. The inventors have found that using the concentrations of fibrinogen and thrombin presented in the Experimental Details Section below, coagulation occurs at about 30 seconds after the fibrinogen and thrombin are combined. However, for other concentrations of fibrinogen and thrombin, the onset of coagulation can be determined by using known coagulation assays.

Suitable oils include but are not limited to vegetable oils (such as corn oil, olive oil, soy oil, and coconut oil), petroleum based oils, silicone oils, and combinations thereof. In the most preferred embodiment, the oil is MCT (medium chain triglycerides) oil preparations.

After the aqueous solution is introduced into the heated oil, the emulsion is then maintained at a temperature of about 50-80° C. and mixed at an appropriate speed until fibrin microbeads comprising extensively cross-linked fibrin(ogen) are obtained in the emulsion. The mixing speed will depend upon the volume of the emulsion, and the desired size of the microbeads. For volumes of 400 mL oil and 100 mL aqueous phase in a 1 L flask, the preferred mixing speed is 300-500 rpm. The emulsion is generally mixed for about 3-9 hours, although the actual time will vary depending upon the temperature, the concentration of the initial reactants and the volume of the emulsion. As discussed above, it is believed that at temperatures of about 65-80° C., the native fibrin(ogen) structure denatures exposing sites for cross-linking by factor XIII, which are not normally cross-linked at ambient temperatures. Such cross-linking occurs during the first phase of the mixing/heating cycle. The heating also serves the purpose of dehydrating the emulsified system (drying process) thereby producing cross-linked fibrin(ogen) particles that do not stick together or coalesce, as such particles do when they possess too much water.

Finally, the extensively cross-linked fibrin microbeads may be isolated from the emulsion using procedures such as centrifugation, filtration, or a combination thereof. The isolated fibrin microbeads may then preferably be washed with solvents such as hexane, acetone and/or ethanol, and then air dried. The microbeads may then be graded to the desired size using commercially available filters or sieves. Preferably, the fibrin microbeads of the present invention are graded to a diameter of about 50-200 microns, although larger or smaller fibrin microbeads may be sized, if desired.

The invention also discloses methods for attachment and growth of cells on FMB in order to form FMB-cell complexes

The fibrinogen used in the present invention may be fibrinogen prepared by fractionation of pooled plasma, or cryoprecipitate obtained from frozen and thawed pooled plasma.

As detailed above, preferred FMB for use in the invention are those having a high degree of cross-linking which are prepared in the absence of exogenous cross-linking agents such as glutaraldehyde, and that are further characterized as immuno-competent and slowly biodegradable, as disclosed for example in US. Pat. No. 6,737,074. Such beads have been disclosed to provide efficient isolation of mesenchymal stem cells and to serve as carriers for matrix-depended cells grown in suspension culture (see e.g. Gorodetsky et al., 2004, ibid), and have also been proposed for use as cellular implants for regenerative medicine (see e.g. Ben-Ari et al., A., Tissue Eng Part A 15, 2537-2546, 2009).

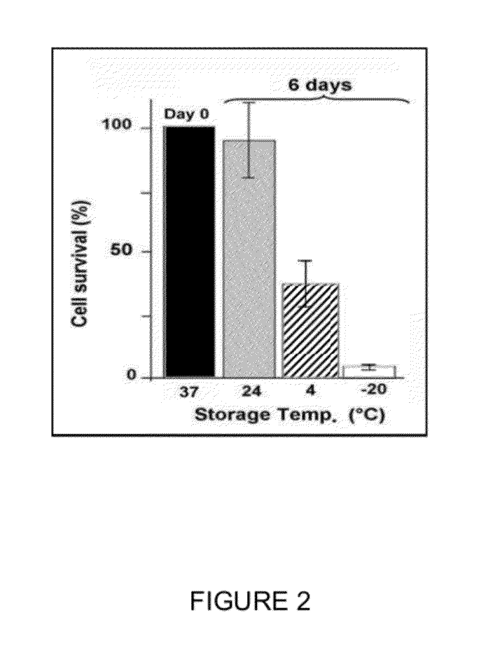

The results disclosed in the Examples herein show that different types of matrix dependent cells, such as mesenchymal stem cells, skin fibroblasts and endothelial cells, when treated according to the principles of the invention, exhibit a high rate of cell survival. More specifically, Example 2 demonstrates that bone marrow-derived mesenchymal stem cells in cell-FMB complexes exhibited a higher rate of cell survival when the complexes were stored at room temperature for 6 days, as compared to the same complexes stored under frozen or cold conditions for the same time period (FIG. 2).

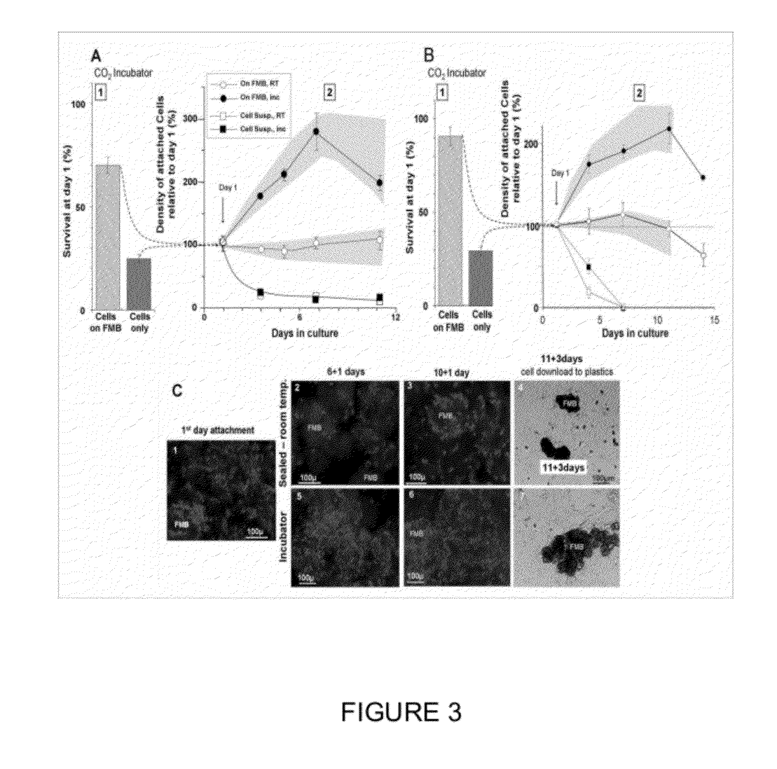

Example 3 demonstrates that multipotent cells from bone marrow and foreskin fibroblasts, when maintained in suspension culture in the absence of fibrin microbeads, showed a poor survival rate after 3 days, whether the cells were maintained at room temperature or in a controlled incubator maintained at 37° C. and 7% CO₂ (FIGS. 3A and 3B). In contrast, the same cells in the form of cell-FMB complexes that were maintained under sealed conditions at room temperature, showed a high survival rate that was close to 100%, even after 10 days of storage (FIGS. 3A and 3B). Furthermore, maintenance of cell-FMB complexes at room temperature was demonstrated to be advantageous over maintenance in an incubator, since the latter procedure was associated with fluctuations in cell density e.g. cell proliferation followed by cell death, whereas the former procedure was associated with a constant cell density (FIGS. 3A and 3B).

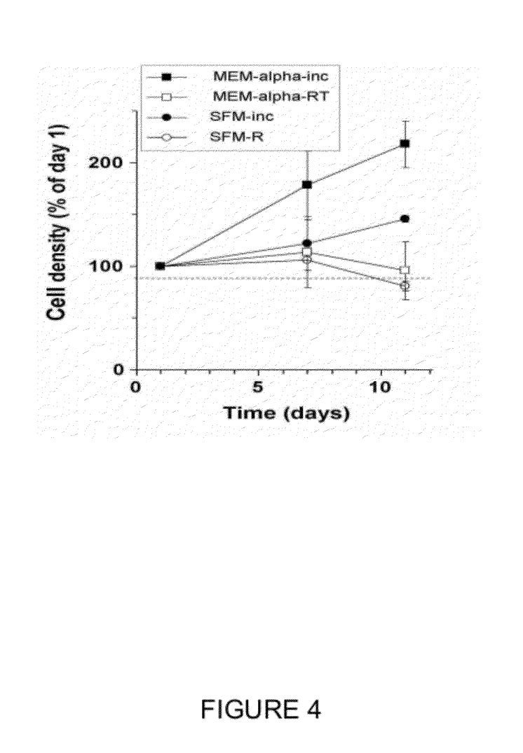

Example 4 demonstrates that the methods of the invention do not particularly require a serum-containing medium, as room temperature storage of FMB-attached cells using either serum-containing medium or serum-free medium was associated with prolonged cell viability (FIG. 4).

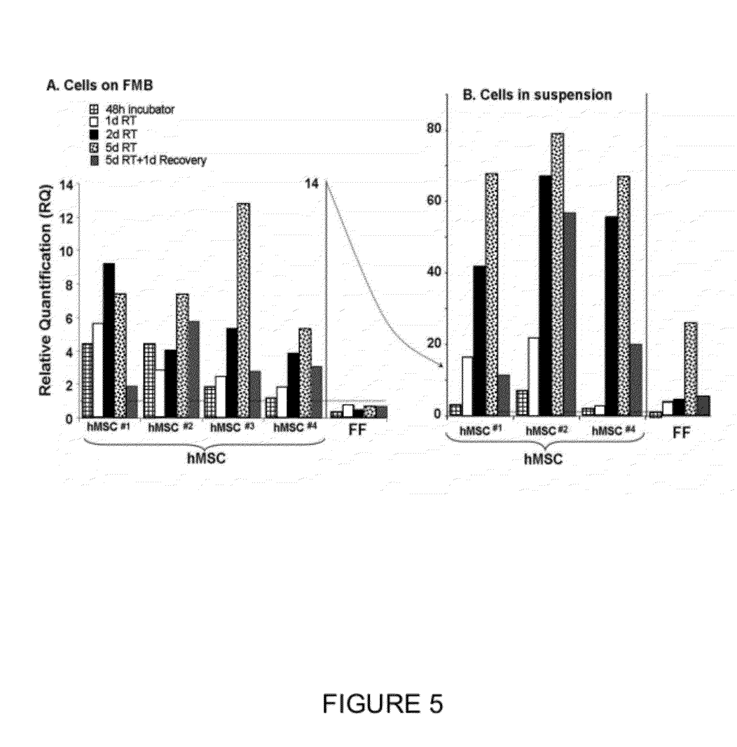

Without being bound by any theory, Example 5 herein demonstrates that the sustained viability of mesenchymal stem cells enabled by the methods of the invention may be associated with up-regulated expression of the gene for hypoxia induced factor HIF1α (FIG. 5).

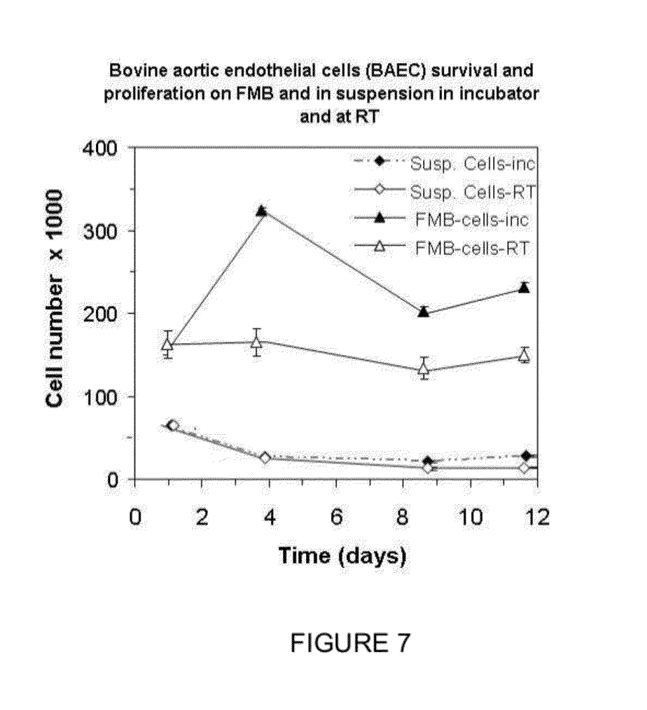

Example 7 herein demonstrates that aortic endothelial cells exhibit a high rate of survival after more than 10 days of storage in the form of cell-FMB complexes under sealed conditions at room temperature (FIG. 7).

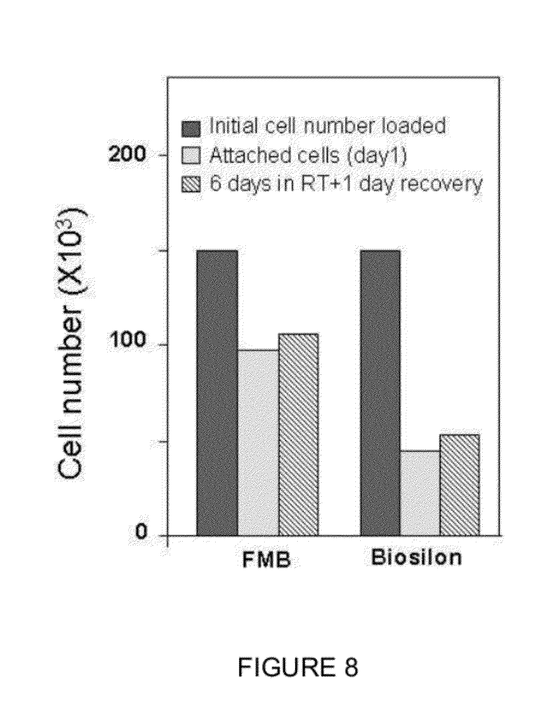

Example 8 herein demonstrates that FMB are superior over polystyrene beads for culture and RT storage of human MSC (FIG. 8).

### DEFINITIONS

As used herein, the terms “matrix dependent cell”, “anchorage-dependent cell” and “adherent cell” interchangeably refer to a cell that requires a solid matrix for growth, such as that of a tissue culture plastic vessel or a microcarrier, and to cells that secret extracellular-matrix when attached to a matrix. The growth surface may be treated or coated e.g. with extracellular matrix components, to enhance cell adhesion. In general, most cells derived from solid tissues are matrix dependent cells.

As used herein, the term “differentiated cell” refers to a cell which is committed to produce a specific specialized cell type and is usually not capable of differentiating into other specialized cell types. Examples of differentiated cells include, without limitation, endothelial cells, smooth muscle cells, striated muscles, skin and interstitial fibroblasts, neuronal cells (e.g. astrocytes, neurons, and oligodendrocytes), cardiac cells, hepatic cells and pancreatic cells.

As used herein, the term “multipotent progenitor cell” refers to a cell which is capable of differentiating, under certain conditions, into a limited number of specialized cell types that derive from its germ line or from other germ lines. Multipotent progenitor cells also have the ability to self-renew for long periods of time. Multipotent progenitor cells have also been termed “adult stem cells” or “mesenchymal stromal cells” to denote cells that are present in tissue of a non-embryonic organism. Multipotent progenitor cells may be obtained for example from bone marrow, umbilical cord blood, peripheral blood, breast, liver, skin, gastrointestinal tract, placenta, and uterus. Multipotent progenitor cells include neuronal stem cells capable of differentiating into neuronal cells, hematopoietic stem cells capable of differentiating into blood cells, mesenchymal stem cells capable of differentiating into bone, cartilage, fat, and muscle, and hepatic stem cells capable of differentiating into hepatocytes.

It is to be understood that the undifferentiated cells according to the present invention are other than totipotent cells. Totipotent cells are the most versatile of the stem cell types and have the potential to give rise to any and all human cells, such as brain, liver, blood or heart cells and may even give rise to an entire functional organism. The first few cell divisions in embryonic development produce more totipotent cells.

As used herein, the terms “mesenchymal stem cells” or “MSC interchangeably refer to plastic-adherent multipotent cells that can differentiate in vitro into lineages including osteoblasts, myocytes, chondrocytes, and adipocytes. MSC are also variously termed “multipotent mesenchymal stromal cells”, and “mesenchymal progenitor cells”, and are found in most tissues and organs. In particular MSC may be derived from bone marrow, growth-factor (such as GCSF) mediated mobilized blood, umbilical cord blood, and adipose tissue.

As used herein, the term “hematopoietic stem cell or “HSC” interchangeably refer to an undifferentiated progenitor cell that gives rise to a succession of mature functional blood cells including red blood cells, different sub-types of white blood cells, and platelet. As used herein, the term “neuronal stem cell (NSC)” refers to an undifferentiated stem cell that resides in the nervous system and generates cells that constitute the nervous system including neurons, astrocytes, and oligodendrocytes.

In the present invention, differentiated cells and multipotent progenitor cells encompass those derived from all animals including humans, monkeys, pigs, horses, cows, sheep, dogs, cats, mice, and rats, and preferably those derived from humans.

As used herein, the term “culture medium” means a medium which enables the growth and survival of mammalian cells in vitro, in particular adult stem cells and mesenchymal cells. A culture medium for use in the invention may include all of the pertinent media typically used in the art. Preferable is a cell culture minimum medium (CCMM), which generally comprises a carbon source, a nitrogen source and trace elements. Examples of a CCMM include, but are not limited to, DMEM (Dulbecco's Modified Eagle's Medium), MEM (Minimal Essential Medium), BME (Basal Medium Eagle), RPMI1640, F-10, F-12, alpha MEM (alpha Minimal Essential Medium), GMEM (Glasgow's Minimal Essential Medium), and IMDM (Iscove's Modified Dulbecco's Medium). A culture medium for use in the invention may further contain one or more of a number of different additives, as is known in the art, for example, an antibiotic, such as penicillin, streptomycin, gentamicin or combinations thereof, amino acids, vitamins, fetal calf serum or a substitute thereof.

As used herein, the term “cultured” in reference to cells means a population of cells that has been grown in the presence of defined culture medium under controlled environmental conditions, typically in an environment maintained at 37° C., and containing about 21% oxygen and about 5-10% CO₂ for mammalian cells. Similarly, the term “culturing” refers to the process of producing an enlarged population of cells by growth of a cell or cells of interest under controlled environmental conditions, typically in an incubator maintained at a set temperature and providing defined concentrations of oxygen and CO₂, and optionally other parameters such as humidity, and agitation in a controlled manner at a set rate.

As used herein, the term “viable” in reference to cells means living cells.

As used herein, the term “cell viability” refers to the percentage of living cells in a given sample, and may be quantitatively assessed by any of a number of methods known in the art.

The terms “preserved viability”, “extended viability”, “prolonged viability”, “extended storage life”, “prolonged maintenance” and “prolonged survival”, and related grammatical terms, are used interchangeably herein, and mean that the percentage of living cells in a cell population or sample thereof following a particular treatment, such as storage under the method of the present invention, is greater (longer, extended or prolonged) relative to a cell population or sample thereof of the same cell type that did not receive that treatment.

The terms “enrichment”, “enrich” and “enriched” in reference to a cell preparation, mean that the processing of an initial cell source, such as bone marrow stroma, results in a cell population having a higher percentage of cells of interest, such as stem cells, in relation to the initial cell source prior to enrichment.

The term “essentially pure” in reference to a population of a particular cell type means that the population contains at least 98%, and preferably at least 99% of the stated cell type.

As used herein, the term “sealed conditions” in reference to a cell preparation means that the cells are present within a container or receptacle such as a tube or flask that is physically closed off from the surrounding environment and generally does not allow liquid or gas exchange with the environment, typically by means of a stopper, plug, valve or screw-cap, as is known in the art. In accordance with the invention, sealed conditions also encompass the case where the container is partially or completely evacuated from atmospheric gases, for example by flushing with gases, prior to sealing.

As used herein, the term “conditions permitting cells to bind” encompasses conditions under which cells in contact with matrices in general and fibrin microbeads in particular adhere thereto and optionally proliferate thereon.

As used herein, the terms “cell-fibrin microbead complexes”, “cell-FMB complexes”, “cells attached to FMB” and “cells loaded onto FMB” are used interchangeably herein to mean fibrin microbeads having cells attached thereto. Typically, the cell-fibrin microbead complexes are obtained upon culturing the fibrin microbeads and the cells.

As used herein, the term “room temperature” means a temperature inside a temperature-controlled environment such as a building, generally in the range of 20 to 26° C.

As used herein, the term “ambient temperature” means the temperature in a particular environment, such as a room or the area surrounding an object in a particular environment, which can vary significantly, depending upon a number of factors, in particular, the presence of climate control, the presence and number of people and/or animals, the presence and type of machinery, and the outside temperature. In general, ambient temperature is within the range of about 16° C. to about 32° C.

As used herein, the term “normoxic” means conditions of normal oxygen concentration which enable optimal cell growth, typically about 21% oxygen for mammalian cells.

As used herein, the term “hypoxic” means sub-normal conditions of oxygen concentration for cell growth for example in the range of 1 to 7% oxygen. Hypoxic conditions may be found in situ in a region of tissue injury, e.g. ischemia, or may be provided and maintained in a controlled environment of cell culture.

As used herein, the term “ambient oxygen” means the oxygen concentration found in a particular environment, such as a room or the area surrounding an object in a particular environment, which can vary significantly, depending upon a number of factors, for example, control of oxygen and other gases, and ventilation in the environment.

### Cell Culture, Isolation and Storage

In a particular embodiment, a method for extending storage life of isolated matrix dependent cells, such as differentiated cells or multipotent cells comprises the steps of:

(i) providing an isolated population of matrix dependent cells;

(ii) culturing the cells of (i) with fibrin microbeads in a culture medium under conditions permitting the cells to bind to the fibrin microbeads; and

(iii) storing the cells bound to the fibrin microbeads obtained in (ii) under sealed conditions in a liquid culture medium at ambient temperature.

A suitable preparation of isolated cells may be one which is enriched for multipotent progenitor cells, typically obtained from a source such as bone marrow, whole peripheral blood, leukopheresis or apheresis products, umbilical cord blood and cell suspensions prepared from tissues or organs.

Multipotent progenitor cells may be isolated using methods of cell culture, expansion and separation known in the art, for example using fibrin microbeads as disclosed by the inventor of the present invention in U.S. Pat. Nos. 6,737,074; and 6,503,731. Alternative methods include those disclosed in U.S. Pat. Nos. 7,592,174; 5,908,782; 5,486,359, and U.S. Patent Application Publication Nos. 2010/0068191; 2009/0124007; and 2010/0297233 among others.

Typical methods for cell enrichment and/or isolation include density step gradients (e.g., Ficoll®, colloidal silica), elutriation, centrifugation, lysis of erythrocytes by hypotonic shock, and various combinations of such methods. For example, purification of stem cells from bone marrow requires removal of erythrocytes and granulocytes, which is often accomplished by Ficoll® density gradient centrifugation, followed by repeated washing steps by conventional centrifugation.

Methods for cell enrichment and/or isolation may also include filtration on various types of filters known in the art for cell separation. For example, tangential flow filtration, also known as cross flow filtration, may be used for enriching stem cells from a heterogenous mixture of bone marrow or blood constituents, as disclosed in U.S. Pat. No. 7,790,039.

Separation of multipotent cells from mixtures may also incorporate a step of absorption to a suitable substrate such as a plastic culture vessel.

In particularly preferred embodiments, fibrin microbeads may be used for isolating cells, as disclosed for example in U.S. Pat. No. 6,737,074 and Gorodetsky et al., 2004 (ibid). Such methods exploit the ability of matrix dependent cells, including differentiated cells and multipotent cells of various types, to attach to and proliferate on fibrin microbeads under standard culture conditions, thereby providing high yields of cell populations.

Accordingly, in some preferred embodiments, the cell preparation of step (i) is obtained by a process comprising (a) culturing cells from a tissue or cell source with fibrin microbeads in a culture medium under conditions permitting the cells to bind to and proliferate on the fibrin microbeads; and optionally (b) separating the cells bound to the fibrin microbeads obtained in (a) so as to obtain a preparation of isolated viable mesenchymal cells. In a particular embodiment, step (b) comprises culturing the mesenchymal cells obtained in (a) on a surface so as to obtain a monolayer, and optionally passaging the cells.

Step (ii) of the method of the invention requires growth of the cells in the presence of fibrin microbeads under suitable conditions so as to form cell-FMB complexes. Accordingly, if the cells were initially grown on FMB and isolated therefrom, a subsequent step of growth on FMB is performed. It is to be explicitly understood however, that steps (i) and (ii) of the disclosed method may be carried out with a single preparation of intact cell-FMB complexes, without any need to separate the cells from the FMB.

Conditions suitable for growth of cells on FMB typically comprise slow rotary or oscillating incubation at 35 to 37° C., in an environment containing about 21% oxygen and between about 5-10% CO₂, with no need of passaging or trypsinization of the cells. Cells may be grown under these conditions until confluence on the beads is attained. The cells may be enzymatically detached from the microbeads for further use, or plated for further use or placed in culture dishes and allowed to passively download from the fibrin microbeads to other matrices on which they may be further cultivated.

Step (iii) comprises storing the cell-FMB complexes obtained in (ii) under sealed conditions at an ambient temperature, for example is in the range of 16 to 32° C. In a particular embodiment, the temperature in (iii) is room temperature i.e. close to 24° C., as is generally found in climate controlled buildings. In a particular embodiment, the temperature in (iii) is ambient temperature. Particularly suitable temperatures for step (iii) are in the range of 18 to 30° C., 20 to 24° C., or in the range of 23 to 26° C.

There is no particular limitation on the volume of cell-FMB complexes stored, although it is generally practical to store aliquots that are suitable for subsequent use, for example for implantation to a human subject or an animal model, or for subsequent expansion or assay. Accordingly, exemplary volumes include those in the range of 20 μA to 2 ml.

Furthermore, the storing in (iii) is generally carried out at a ratio of (cell-FMB complexes:liquid culture medium) in the range from 1:5 to 1:50 (v/v). Storage of the cell-FMB complexes in (iii) may be carried out for at least 3 days, and may be readily carried out for longer periods, such as at least 6 days. In additional embodiments, the storing in (iii) is carried out for up to 21 days, or up to 28 days.

The cell-FMB complexes are conveniently stored in small cap-fitted tubes such as those intended for storing cells in a frozen state, which are commercially available from a number of manufacturers e.g. CyroTubes® (Nunc).

For storage of cell-FMB complexes, a small volume of the complexes is transferred to the container for use, and the empty volume is filled with a suitable culture medium, followed by tightly stoppering the tube with a liquid tight closure. In some cases, air may be evacuated prior to or following the filling step, typically by flushing the tube with a gas mixture such as N₂ and CO₂. In a particular embodiment, the storing in (iii) is carried out under normoxic conditions following exposure to ambient oxygen conditions.

The storing in (iii) is conveniently carried out in the absence of an incubator and/or agitating conditions.

Following the storage period and prior to use, the cells may be subjected to a recovery period which comprises incubating the cell-FMB complexes at 37° C. Such a recovery period may be carried out for a period of 10 to 30 hours, or for a period of up to 24 hours. Typically this step is carried out in an incubator, i.e. an environment containing about 21% oxygen and between about 5-10% CO₂.

The liquid culture medium used for growth, expansion and/or storage of matrix dependent cells may be a serum-containing medium or a serum-free medium.

The term “serum-free medium” may comprise cell culture media which is free of a mixture of human or animal serum as is found in fetal calf serum. Alternately, a serum-free medium may contain one or more isolated and purified serum proteins such as serum albumin. Serum-free media suitable for growth of mesenchymal stem cells are known in the art. For example, a medium comprising fibroblast growth factor-2, Leukemia Inhibitory Factor and Stem Cell Factor, sodium pantotenate biotin and selenium is known. Also known is a medium comprising a minimum essential medium; serum albumin; an iron source; insulin or an insulin-like growth factor; glutamine; and a mitogen selected from the group consisting of platelet derived growth factor and serotonin. Other known media include, but are not limited to, a medium comprising an additive formed by alcohol hydrolysis of an intact phosphoglyceride ester defining a substituted glycerol; a medium containing 10 to 800 ng/ml tissue inhibitor of metalloproteinases; a medium containing a specific growth factor and at least one phospholipid; and a medium comprising various combinations of growth factors including bFGF, TGF-beta, and EGF.

The practice of the present invention will employ, unless otherwise indicated, conventional techniques of chemistry, molecular biology, microbiology, recombinant DNA and immunology, which are within the capabilities of a person of ordinary skill in the art. See, for example, J. Sambrook, E. F. Fritsch, and T. Maniatis, 1989, Molecular Cloning: A Laboratory Manual, Second Edition, Books 1-3, Cold Spring Harbor Laboratory Press.

### Multipotent Progenitor Cells

Isolated multipotent progenitor cells for use in the invention include those termed “mesenchymal stem cells” (also referred to herein as “MSC”), such as those obtained from bone marrow stroma and umbilical cord blood, which have the ability to differentiate in vitro into different cell types, in particular chondrocytes, osteoblasts, adipocytes and myocytes. In vitro studies have demonstrated the capability of MSC to differentiate into muscle, neuronal-like precursors, cardiomyocytes and possibly other cell types. In addition, MSC have been disclosed to provide effective feeder layers for expansion of hematopoietic stem cells.

Studies with a variety of animal models have shown that MSC may be useful in enhancing the repair or regeneration of damaged bone, cartilage, meniscus or myocardial tissues spinal cord injury.

MSC are also variously termed “multipotent mesenchymal stromal cells”, “mesenchymal progenitor cells” and “nonhematopoietic stem cells”.

Other sources of matrix dependent proliferating cells for use in the invention include skin fibroblasts, myofibroblasts, smooth muscle cells, fibrocytes, endothelial cells, amnion mesenchymal cells, chorion mesenchymal cells, adipose tissue, periosteum and transgene-activated mesenchymal cells.

The matrix dependent progenitors of proliferating cells may be from a human or from a non-human mammal.

In a particular embodiment, the mesenchymal progenitor cells are adult human mesenchymal cells.

### Fibrin Microbeads

FMB having a high density of cells attached thereto (also referred to herein as “cell-loaded FMB”) have been produced by growth of various cell types in three dimensional (3D) slow rotating suspension cultures, and have been proposed for various applications in cell-based regenerative medicine (e.g. Gorodetsky et al., 2004, ibid; Rivkin et al., 2007, ibid). The differential binding to FMB of matrix-dependent cell types, such as those from mesodermal origin, enables their use as a highly efficient tool for isolation of mesenchymal stem cells (MSC) from different sources (e.g. Ben-Ari et al., ibid). It has been proposed that the cell attachment to FMB is aided by conserved sequences at the C-termini of beta- and gamma-fibrin chains which are exposed on the FMB surface (Gorodetsky et al., Exp Cell Res 287, 116-129, 2003). MSC attached onto FMB can efficiently expand in 3D culture without the need for passaging and trypsinization. Upon appropriate induction, MSC-loaded FMB can be induced, both in vitro and in vivo, to differentiate into various cell types of interest, such as osteoblasts and chondroblasts. Such cell-FMB materials have been utilized in the formation of bone and cartilage-like tissue constructs (e.g. Ben-Ari et al., ibid and Shainer et al., Regen Med 5, 255-265, 2010). Adult differentiated cells, such as human foreskin fibroblasts (FF) are also capable of growth on FMB and may thus serve as constructs for implantation (Gorodetsky et al., J Invest Dermatol. 112, 866-872, 1999).

The fibrin microbeads for use in the invention are preferably those as described in U.S. Pat. Nos. 6,737,074; 6,503,731 and 6,150,505.

An exemplary method for producing fibrin microbeads includes the following procedures.

First, an aqueous solution comprising fibrinogen, thrombin and factor XIII is prepared. This solution may be prepared by combining fibrinogen containing endogenous factor XIII with thrombin, by combining cryoprecipitate containing endogenous fibrinogen and endogenous factor XIII with thrombin, or by combining fibrinogen, factor XIII and thrombin individually into an aqueous solution. Equivalent proteases such as snake venom proteases (e.g. reptilase) may be used as an alternative to thrombin. The ratio of fibrinogen:thrombin:factor XIII in the aqueous solution is preferably in the range 5-100 mg/mL:1-100 U/mL:1-50 U/mL, and preferably 20-40 mg/mL:5-10 U/mL:2-20 U/mL. In addition to these proteins, the aqueous solution also may contain fibronectin and other blood-derived proteins that may be present in the fibrinogen and cryoprecipitate starting materials. The fibrin microbeads may additional contain one or more bioactive agents, which can be added into the fibrinogen or thrombin solutions prior to their mixing, or directly to the aqueous solution.

Next, prior to the onset of coagulation, the aqueous solution is introduced into an oil heated to a temperature in the range of about 50-80° C. to form an emulsion. A hydrophobic organic solvent such as isooctane also may be included in the oil. Typically, coagulation occurs at about 30 seconds after the fibrinogen and thrombin are combined. However, for other concentrations of fibrinogen and thrombin, the onset of coagulation can be determined by using known coagulation assays.

Suitable oils include but are not limited to vegetable oils (such as corn oil, olive oil, soy oil, MCT and coconut oil), petroleum based oils, silicone oils, and combinations thereof. Vegetable based oils are preferred because they can be metabolized by cells and may provide nutrients to the cells. In a preferred embodiment, the oil is corn oil.

After the aqueous solution is introduced into the heated oil, the emulsion is then maintained at a temperature of about 65-80° C. and mixed at an appropriate speed until fibrin microbeads comprising extensively cross-linked fibrin are obtained in the emulsion. The mixing speed will depend upon the volume of the emulsion, and the desired size of the microbeads. For volumes of 400 mL oil and 100 mL aqueous phase in a 1 L flask, a preferred mixing speed is 100-500 rpm in the early phase of up to 1 hr and 100-200 rpm later. The emulsion is generally mixed for about 3-9 hours, although the actual time will vary depending upon the exact temperature along the process, the concentration of the initial reactants and the volume of the emulsion. It is believed that at temperatures of about 60-80° C., the native fibrin structure denatures exposing sites for cross-linking by factor XIII which are not normally cross-linked at ambient temperatures Such cross-linking occurs during the first phase of the mixing/heating cycle. The heating also serves the purpose of dehydrating the emulsified system (drying process) thereby by dehydrothermal non-enzymatic crosslinking, producing cross-linked fibrin particles that do not stick together or coalesce, as such particles do when they possess too much water.

Finally, the extensively cross-linked fibrin microbeads may be isolated from the emulsion using procedures such as centrifugation, filtration, or a combination thereof. The isolated fibrin microbeads may then preferably be washed with solvents such as hexane, acetone and/or ethanol, and then air dried. The microbeads may then be graded to the desired size using commercially available filters or sieves. Preferably, the fibrin microbeads of the present invention are graded to a suitable diameter, typically in the range of 20 to 500 nm, preferably about 50-200 nm or 50 to 100 nm, although larger or smaller fibrin microbeads may be size selected.

The fibrin microbeads may be manufactured to further contain additional biodegradable polymers, for example, homo- or copolymers of glycolide, such as L-lactide, DL-lactide, meso-lactide (polylactide, PLA), e-caprolactone (polycapro lactone, PCL), 1,4-dioxane-2-one, d-valerolactone, B-butyrolactone, g-butyrolactone, e-decalactone, 1,4-dioxepane-2-one, 1,5-dioxepan-2-one, 1,5,8,12-tetraoxacyclotetradecane-7-14-dione, 1,5-dioxepane-2-one, 6,6-dimethyl-1,4-dioxane-2-one, and trimethylene carbonate; block-copolymers of mono- or difunctional polyethylene glycol; block copolymers of mono- or difunctional polyalkylene glycol; blends of the above mentioned polymers; polyanhydrides and polyorthoesters; such as copolymers of poly(D,L-lactide-co-glycolide) (PLGA), MPEG-PLGA (methoxypolyethyleneglycol)-poly(D,L-lactide-co-glycolide) poly(L-lactic acid (PLLA), poly(DL-lactic acid (PLA), poly(DL-lactic-co-glycolic acid) (PLGA), polyorthoesters, polyanhydrides, polyphosphazenes, polycaprolactones, polyhydroxyalkanoates, biodegradable polyurethanes, polyanhydride-co-imides, polypropylene fumarates, polydiaxonane, polysaccharides, collagen, silk, chitosan, and celluloses

Additional optional components that can be incorporated into the fibrin microbeads include glycosaminoglycans, proteoglycans and proteins, including those found in the extracellular matrix. Example of such additional components include chondroitin sulfate, hyaluronic acid, heparin sulfate, heparan sulfate, dermatan sulfate, elastin, collagen, such as collagen type I and/or type II, gelatin and aggrecan.

Additional optional components that can be incorporated into the fibrin microbeads include growth factors, such as insulin-like growth factor 1 (IGF-1); transforming growth factors (TGFs), such as TGF-alpha or TGF-beta; fibroblast growth factors (FGFs), such as FGF-1 or FGF-2 or bone morphogenic protein (BMP).

Other types of fibrin microbeads may be used in the invention, including those disclosed in Senderoff et al. (ibid).

### Therapeutic Applications

Multipotent progenitor cells produced according to the invention may be used in various therapeutic applications, in particular for implantation. The implanted cells may be those that remain attached to the fibrin microbeads, or that are separated therefrom.

The invention may be used for treating a number of defects and diseases, in particular, cartilage defects, bone defects, bone diseases, osteoarthritis, osteoporosis, spinal cord injury, periodontal disease, myocardial infarction. The invention further provides methods for bone, cartilage or tissue reconstruction, comprising administering to a subject in need thereof a cell preparation, or a composition comprising a cell preparation of the invention. Alternately, the methods may comprise implanting an implantable device comprising a cell preparation of the invention, to a subject in need thereof.

Administration may be carried out by injection, intravenous delivery or direct instillation

In particular embodiments, an implantable device comprises a cell preparation of the invention. In particular embodiments, an implantable device comprising a cell preparation of the invention is for use in the restoration, the reconstruction, and/or the replacement of tissues and/or organs.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Materials and Methods

The following procedures were used in the Examples that follow.

### Fibrin Microbeads (FMB).

FMB were prepared as described in Gorodetsky et al., (2004, ibid), using paste 2 fibrin-enriched fractionated plasma obtained from NABI Biopharmaceuticals (Rockville, Md.). The raw fibrin rich materials were further purified by sedimentation in 10% ethanol at 4° C., followed by reconstitution of the sediment in Tris buffer to yield a solution with 60-80 mg/ml soluble clottable protein. The obtained fibrinogen solution was treated with ˜1 Unit/ml thrombin (Omrix, Israel) with a final concentration of about 3 mM, and was then immediately added to rapidly stirring and heated (80° C.) MCT mineral oil to form an emulsion with small droplets from which very dense beads were formed within 7-9 hours, as previously described in Gorodetsky et al, 2004, ibid. The resulting solid FMB were cleaned from oil, dried and sieved and the main fraction having a size range of 105-180 μm was used. Prior to use, the beads were sterilized by incubation for 12 hrs in 70% ethanol, which was then replaced by medium in which the FMB were re-hydrated for a few hours.

### Foreskin Fibroblasts (FF).

Foreskin fibroblasts (FF) were prepared from discard donations of circumcision-removed tissue from normal 7 day old human infants, or commercial preparations of isolated FF (Forticell Biosciences) were used. For isolation of FF from tissue, the source material (˜800 mg) was harvested into medium containing DMEM/10% fetal calf serum (FCS; GIBCO®)/10% penicillin-streptomycin (pen-strep) and incubated for up to 1.5 hr at room temperature. The sample was then cleaned of fat, stripped of epidermal cells and chopped by scalpel and scissors in sterile conditions into ˜1-3 mm pieces. The pieces were rinsed several times in full medium (DMEM/10% FCS/1% pen-strep/1% glutamine/1% non-essential amino acids/1% vitamin solution), all from Biological Industries, Israel). About 10 pieces were transferred to each plastic culture flask, and distributed evenly on the bottom surface of the flask. Flasks were incubated in vertical position overnight in a 37° C., 7% CO₂ incubator. The next day 2 ml full medium was carefully added to each flask so that the pieces stayed attached to the surface. Medium was replaced twice weekly over a period of 2-4 weeks until the cells formed a monolayer (also referred to herein as “downloaded cells”). Cells reaching confluence were split by trypsinization. Generally, after 2-3 passages, the cultures stabilized into homogenous fibroblast-shaped cells and were then used for experimental purposes. The isolated expanded cells from each source were tested to have normal chromosomal karyotype. Commercial FF were cultured in flasks in the presence of full medium and passaged as described above.

Separation and Growth of Human MSC (hMSC) from Bone Marrow.

Samples of bone marrow from 4 different normal adult young volunteers were purchased from Lonza (UCLA). Bone marrow-derived human mesenchymal stem cells (hMSC) were isolated by an FMB-based adhesion protocol modified from that previously used for mouse and rat MSC. Essentially, the source bone marrow was diluted 1:3 in full hMSC medium (MEM-alpha/1% pen-strep, 1% glutamine/20% GIBCO® FCS). MEM-alpha, pen-strep and glutamine were from Biological Industries, Israel, and GIBCO® FCS was from Invitrogen (Carlsbad, Calif., U.S.A.). Diluted bone marrow was combined with FMB at a ratio 10 ml/150 μl of prewetted FMB in 50 ml tubes fitted with filter-caps for gas exchange and incubated for 48 hours with rotation. After 48 hours, the medium was changed to remove non-attached cells, and FMB with isolated mesenchymal cells attached thereto (also referred to herein as “cell-loaded FMB”) were transferred to plastic plates to enable “downloading” of the cells to the plate surface. Detached FMB were removed by washes with medium.

The cells attached to the plastic surface are also referred to herein as “downloaded” cells. An essentially pure population of isolated hMSC obtained after 2-3 passages was found by FACS staining to be positive for the human mesenchymal stem cell markers SCA1, CD105, CD106, CD90 and CD44.

An exemplary set of up of cell culture on FMB is shown in FIG. 1. In this set up a rotator in the CO₂ incubator is used for culturing the cells on FMB (FIG. 1A) in tubes closed with perforated covers for gas exchange (FIG. 1C). An electron scanning microscopy image exhibiting cells attached to FMB is shown in FIG. 1B. For storage, the cells, attached to FMB, are transferred in cryotubes topped up with culture medium and sealed to exclude air (FIG. 1D).

Nutristem™ serum free medium (SFM; Biological Industries, Israel) was used in place of FCS-containing medium for some experiments with hMSC.

### Bovine Aortic Endothelial Cells (BAEC)

BAEC were separated from aortas of slaughtered cows. The internal volume of each aorta samples was rinsed numerous times with sterile phosphate buffered saline with adequate additives and antibiotics. Then the endothelial in internal layer of the vessel was scraped carefully with a sterile applicator, so as to avoid penetrating the smooth muscle layer. The layer of cell was then collected and dispersed on plastic culture dishes. The cells were placed on small plastic dishes and the cell colonies that emerged from the samples that adhered to the plastics were harvested and transferred to larger plates for further cultivation in adequate medium such as low glucose DMEM (Biological Industries) to establish the culture of BAEC.

### Extended Storage of Cell-Loaded FMB

Cultured cells were loaded onto FMB by trypsinizing them away from the plastic surface on which they were grown and adding the cell suspension to an adequate volume of FMB in a sterile polyethylene tube with air exchange that was placed within the CO₂ incubator. One to 2 days later the non-attached cells were discarded by exchanging the medium, leaving only cells adhered to FMB. Cell-loaded FMB (˜50 μl) were transferred into cryovials (CryoTubes™; Nunc). The tubes were filled with adequate medium supplemented with FCS or with SFM (volume˜800 μl). Then the tubes were tightly sealed, with care taken to exclude air bubbles.

One series of experiments was directed at determining the effect on cell survival of different temperatures under sealed conditions for one week Another series of experiments was directed at following survival of cells maintained in sealed conditions at ambient conditions for different time intervals versus cells maintained at 37° C. in a CO₂ incubator. In these experiments, sealed cryotubes containing cell-loaded FMB or cells in suspension were maintained at RT for different time intervals, with 3 samples for each time interval. At the time point of interest, the tubes were opened and the cells were transferred to a CO₂ incubator for a recovery period of 24 hrs. As a control, trypsinized cells were kept in suspension in similar conditions. The number of surviving cells was assessed by MTS assay.

### MTS Assay for Cell Density, Survival and Proliferation.

The number of cells loaded on FMB was assayed by MTS assay using CellTitre 96® aqueous assay (Promega, Madison, Wis.) which monitors the total number of living cells in the sample. The assay was modified for FMB, as previously described. Briefly, tubes containing cell-loaded FMB or matching negative controls lacing cells were tested in triplicate. At the end of MTS color development, samples of the supernatant were transferred to 96 well plates and monitored for absorbance at OD₄₉₂ by a computerized plate reader (Tecan Sunrise, Austria). Values for OD₄₉₂ were converted into numbers of viable cells using a calibration curve obtained from multi-well plates containing known cell numbers.

### Microscopy.

Microscope images were obtained with a DS-R1 color camera for fluorescence with DS-L2 controller mounted on an Eclipse TE200 inverted microscope with Nomarsky optics plus fluorescence set-up (all from Nikon, Japan).

### Nuclei Staining to Evaluate Cell Density on FMB.

Samples of cell loaded FMB were fixed with 70% ethanol or glutaraldehyde or formalin. Then the cell-loaded FMB was treated with 2.5 μl of 50 μg/ml propidium iodide solution (Sigma, Israel) for 5 minutes in the dark. The staining solution was removed and the sample was mounted on a slide by mounting solution (Sigma, Israel). The red-stained nuclei of the cells were visualized by fluorescence microscopy.

RNA extraction and Real time PCR.

RNA was extracted by a modified TRIzol® based protocol (Invitrogen, U.S.). Samples of cells loaded on FMB were placed in small Eppendorf tubes with secured lock with 1 ml TRIzol®. Following vigorous shaking with Small metal steel balls for 30 seconds a fully homogenized solution was obtained. Then 200 μl of chloroform was added, and mixed well. Following 10 min incubation at 4° C., the samples were centrifuged at 12,000 g for 15 minutes. The RNA in the colorless upper aqueous phase was transferred to fresh 1.5 ml eppendorf tube and precipitated from the aqueous phase by mixing with 0.5 ml 100% isopropyl alcohol. Following 10 min incubation of the samples at RT and centrifugation at 12,000 g for 15 minutes at 4° C., the precipitated RNA pellet was collected and washed with >1 ml 70% and 100% ethanol at 4° C. The RNA pellet was air dried for 3 min, then dissolved in purified RNase-free water (20-50 μl) and stored at −80° C.

The RNA integrity was confirmed by electrophoresis on ethidium bromide-stained 1% agarose gel. RNA concentration was determined by NanoDrop™ (Thermo Scientific, Wilmington, Del.). A sample of RNA (1 μg) was amplified with the High Capacity cDNA Reverse Transcription Kit (Applied Biosystems, U.S.) to generate 20 μl of cDNA. A 1-2 μl sample of the cDNA was then quantified by real time PCR Real time PCR using the 7900HT FAST™ Real-Time PCR System (Applied Biosystems, U.S.). TaqMan® Gene Expression Master Mix and TaqMan® was used with Gene Expression Assays (Applied Biosystems, U.S.). The quantity of PCR product generated from amplification of the gene was standardized using human Pactin house keeping gene (Hs 99999903_A1) and the probe for HIF-1α was Hs 00936366_A1.

### Example 1

### FMB Isolation of hMSC and Culture in 3D Conditions

hMSC were isolated from bone marrow of 4 donors with the use of the FMB based adhesion protocol (FIG. 1A). This protocol is documented to provide higher yields of MSC with improved purity. The isolated hMSC were downloaded from the FMB to plastic plates after 4 days in culture, and expanded for 2-3 passages. At this stage, nearly 95% of the isolated cells were shown by FACS analysis to express CD44, CD29, SCA1, and less than 5% were CD45 and CD11 positive. The purified expanded hMSC were then re-loaded on FMB by growth in slowly rotating suspension cultures (FIGS. 1A-C).

### Example 2

### Cell Survival at Different Temperatures

hMSC loaded on FMB, as described in Example 1, were sealed in cryotubes topped up with growth medium and incubated for 6 days at one of the following temperature conditions: frozen (−20° C.; FIG. 2, white shaded bars), cold (4° C.; FIG. 2, diagonally shaded bars), room temperature (about 24° C.; FIG. 2, gray shaded bars).

Then the cells were allowed to recover by incubation in a CO₂ incubator for 24 hrs and the number of surviving cells was assessed by the MTS assay. As shown in FIG. 2 (results are presented relative to the initial cell number observed upon removal from the incubator (37° C. and 7% CO₂; FIG. 2, black shaded bars)), only a small percentage (>4%) of the frozen cells and less than 40% of the cells stored at 4° C. survived and showed recovery, while 95% of the cells stored at RT exhibited survival and recovery.

The results strongly indicate that storage of cell-FMB complexes in the range of room temperature is advantageous for promoting cell survival, as compared to storage at freezing or cold temperatures.

### Example 3

### Cell Survival Following Growth on FMB as a Function of Time

In order to assess cell survival on FMB as a function of time, hMSC from 3 different donors were loaded onto FMB as described in Example 1, sealed in cryotubes and maintained at RT for different time periods. Prior to assessment of cell survival by the MTS assay, the cells were allowed to recover for 24 hrs under optimal conditions i.e. rotating incubation in a 37° C., 7% CO₂ incubator. In parallel, trypsinized cells were maintained in suspension for the same time periods.

The results of a representative experiment of hMSC from a single donor (4 replicates) are shown in FIG. 3. Cells were grown onto FMB (FIG. 3A-1, circle) or in suspension (FIG. 3B-1, squares) at 37° C. in a CO₂ incubator and cell survival (number of residual living cells) was assessed after 24 hours. Subsequently, cells of the systems shown in FIGS. 3A-1 and 3B-1 were maintained either at 37° C. in a CO₂ incubator (black circles and squares), or in sealed conditions at RT (white circles and squares) over a period of 10 days, and the results of cell survival at different time points were normalized against the cell number at day 1 (FIGS. 3A-2 and 3B-2). As shown in FIG. 3A-1, of the cells maintained in suspension (black circles and squares), without FMB, the majority (75%) died within the first day. In contrast, of the cells attached to FMB (white circles and squares), ˜80% survived at the first day.

The data in FIGS. 3A and 3B represent 3 replicates of cells from a single donor. The shaded areas represent the range of the collective results obtained from cells of 3 donors of each cell type.

The hMSC surviving at day 1, both those grown in suspension and those grown attached to FMB served as a reference (100%) for the subsequent follow up of survival of cells maintained in sealed tubes at room temperature, or under normal culture conditions in a CO₂ incubator at 37° C. (FIG. 3A-2). As shown, the FMB-loaded hMSC maintained at RT (white circles) showed a survival rate of 100% or greater at the 10 day time point, and no decrease in the cell number was observed. The hMSC-loaded FMB maintained in the incubator (black circles) showed continued proliferation of the cells until confluence was reached, after which there was a sharp drop in cell number following day 7. The results obtained with hMSC from the 3 tested donors were similar, and are collectively indicated by the shaded plot areas (FIG. 3A-2).

In contrast, hMSC that were maintained in suspension culture without FMB, either at RT (white squares) or at 37° C. in the CO₂ incubator (black squares), showed poor survival with only about 25% viability by the day 3 time point and about 10% viability by the 10 day time point (FIG. 3A-2).

Results obtained with human normal foreskin fibroblasts (FF) were similar to those observed with hMSC. As shown in FIG. 3B-1, of the cells maintained in suspension without FMB, the majority (75%) died within the first day. In contrast, of the cells attached to FMB, ˜90% survived at the first day.

In addition, FF-loaded FMB which were stored under sealed conditions at RT, showed a cell survival rate of 100% or greater by the 10 day time point, with no decrease in the cell number (FIG. 3B-2; white circles). In contrast, FF that were maintained in suspension culture without FMB, either at RT (white squares) or at 37° C. in the CO₂ incubator (black squares), showed poor survival with only about 20% and 50% viability respectively by the day 3 time point, and 0% viability by the 7 day time point (FIG. 3B-2).

Cell-loaded FMB were semi-quantitatively assessed for cell density by nuclei staining at different time points of storage. Cells examined were maintained at RT (FIG. 3C panels 1-3) or in the incubator (FIG. 3C panels 1, 5 and 6) for the time intervals indicated. At day 12 the cell-FMB complexes were placed on a plastic surface and functional living cells were downloaded (FIG. 3C panels 4 and 7). As shown in FIG. 3C, panels 1-3, hMSC attached to FMB and maintained under sealed conditions at RT exhibited a constant density of cells from the first day of attachment through to the 10^{th} day of storage. In contrast, hMSC attached to FMB and maintained in the incubator, continued to proliferate (FIG. 3C, panels 5-6), consistent with the results shown in FIG. 3-A2.

At day 12, the cell-loaded FMB from both groups were transferred to plastic surfaces and downloading of functional viable cells from both the RT-stored group and the incubator-stored group was evident (FIG. 3C, panels 4 and 7 respectively).

The results disclosed herein show that different types of mesenchymal cells, when attached to FMB and stored for prolonged periods i.e. at least 10 days, under sealed conditions at RT, exhibit a high rate of cell survival. In contrast, mesenchymal cells maintained in suspension culture in the absence of FMB, show a poor survival rate, whether maintained at RT or in a 37° C., CO₂ incubator. Furthermore, for long-term storage of FMB-attached cells, maintenance at RT under sealed conditions is advantageous over maintenance in an incubator, since the latter is associated with fluctuations in cell density e.g. cell proliferation followed by cell death, whereas the former is associated with a constant cell density.

### Example 4

### Effect of Serum Free Medium on Survival of FMB-Attached Cells

To examine the possible effect of the inclusion of FCS in the medium on the RT-sustained survival of hMSC attached to FMB, hMSC from one of the bone marrow sources was maintained under sealed conditions with serum-free medium (SFM) for stem cells (Nutristem™). As shown in FIG. 4, no significant difference was seen in the RT-sustained survival of FMB-attached hMSC between the cultures maintained in FCS-containing medium (white squares) versus those maintained in SFM (white circles). In contrast, FMB-attached hMSC cultures maintained in an incubator showed a higher proliferation rate in FCS-containing medium (black squares) versus those in SFM (black circles). The observed results suggest that RT-sustained survival of hMSC attached to FMB is not dependent on the present of FCS in the medium.

### Example 5

### Involvement of hypoxia induced factor 1α (HIF1α)

The expression of HIF1α was examined in a study directed to identifying regulatory mechanisms that may be associated with the RT-sustained survival of FMB-attached mesenchymal cells. Real-time qPCR was performed on RNA collected from samples of hMSC (from different donors), attached to FMB and stored at RT for a period of 5 days with a 24 hour recovery period. In parallel, RNA from cells surviving upon maintenance in suspension in the absence of FMB was obtained and examined for HIF1α expression. The reference control used was HIF1α expression in cells grown in monolayer.

When attached to FMB and maintained under sealed conditions at RT, hMSC from all the sources tested exhibited time-dependent elevation of HIF1α expression (FIG. 5A). As shown, over the course of the 5 day storage period (FIG. 5A: 1 day—white shaded bars; 2 days—black shaded bars; 5 days—dots shaded bars), HIF1α expression increased 8- to 12-fold, compared to control “incubator” levels (48 hours at 37° C. in the CO₂ incubator; FIG. 5, grid shaded bars). Upon recovery (FIG. 5: gray shaded bars), HIF1α expression by hMSC was significantly decreased in all samples, compared to the maximal levels achieved, and in 2 of the cases (#1 and #3), the recovery levels were less than or similar to the control “incubator” levels.

No significant increase in HIF1α expression was observed in FF attached to FMB that were maintained under the same conditions (FIG. 5A; FF).

For comparison, HIF1α expression was examined in hMSC grown in suspension in the absence of FMB, and maintained at RT. Only a small proportion of such hMSC survived, possibly due to formation of small cellular aggregates. The surviving cells analyzed showed significantly higher levels of HIF1α expression (by almost one order of magnitude), as compared to the FMB-attached cells at the same time points (FIG. 5B vs. Fib. 5A). Furthermore, the observed time-dependent elevation of HIF1α expression corresponded to a 40- to 80-fold increase, compared to control “incubator” levels. Recovery of the cells had a significant effect on reversing the elevation of HIF1α expression.

### Example 6

### Assessment of Total Extractable Cellular RNA

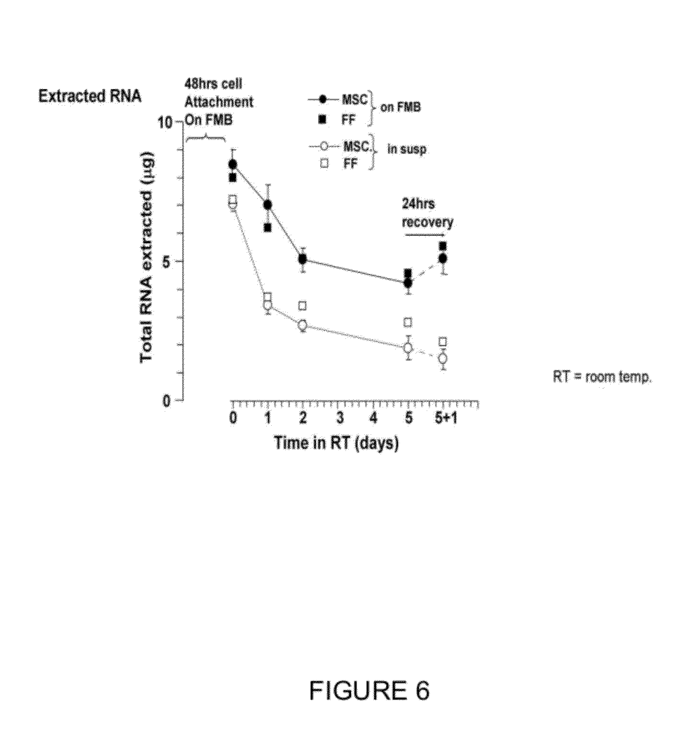

Assessment of total extractable cellular RNA showed decreases in RNA over time, in both of hMSC and FF attached to FMB when maintained at RT (FIG. 6; black circles and black squares, respectively). In particular, the levels sharply decreased in the first two days, while the rate of decrease leveled off considerably between 2 and 5 days. However, the total amount significantly increased upon recovery (FIG. 6).

In comparison, the rate of RNA decrease was much more rapid over the first day, in both of hMSC and FF grown in suspension in the absence of FMB (FIG. 6; white circles and white squares, respectively). Furthermore, no restorative increase in the amount of RNA was seen due to the recovery period, in contrast to the FMB-grown cultures.

These results suggest that mesenchymal cells attached onto FMB are capable of recovering metabolic processes that might be impaired during storage at RT, as indicated by RNA content. In contrast, mesenchymal cells grown in the absence of FMB do not display such recovery following storage at RT.

### Example 7

### Survival of Bovine Aortic Endothelial Cells (BAEC) Following Growth on FMB

BAEC attached to FMB or grown in suspension culture were assessed for survival at different time points following storage at RT, or 37° C. in a CO₂ incubator, essentially as described in Example 3. Specifically, cells were grown onto FMB or in suspension (Susp.) at 37° C. in a CO₂ incubator for 24 hours. Subsequently, cells were maintained either at 37° C. in a CO₂ incubator, or in sealed conditions at RT over a period of 12 days, and number of surviving cells was assessed at each time point. The graph shows cell number of cultures grown on FMB followed by storage at RT (white triangles) or at 37° C. in the incubator (black triangles); and cultures grown in suspension followed by storage at RT (white diamonds) or at 37° C. in the incubator (black diamonds).

As shown in FIG. 7, the FMB-loaded BAEC maintained at RT (white triangles) showed a survival rate close to 100% at the 12 day time point, since only a negligible decrease in the cell number was observed. The BAEC-loaded FMB maintained in the incubator (black triangles) showed continued proliferation of the cells until day 4, after which there was a sharp drop in cell number until day 9, followed by an increase.

In contrast, BAEC that were maintained in suspension culture without FMB, either at RT (white diamonds) or at 37° C. (black diamonds), exhibited a lower initial number of cells that survived in the suspension (i.e. about half of that of the FMB adhered cells), and thereafter showed poor survival with 25% or less of surviving cells by the day 12 time point for cells stored at RT. The results demonstrate that FMB beads are a suitable matrix for attachment and storage of differentiated cells, as well as multipotent progenitors of various types.

### Example 8

### Survival of Human MSC Following Growth on FMB or Polystyrene Beads

Human MSC (˜1.5×10⁴ cells) were attached to FMB or to commercially available polystyrene beads (160-300 mm) for adherent cell culture (Biosilon®) by growth at 37° C. in a CO₂ incubator for 24 hours (attachment period). Subsequently, both types of MSC-loaded beads were stored at RT for 6 days (storage period), followed by incubation at 37° C. for one day (recovery period). As shown in FIG. 8, while the initial number of cells loaded to each type of beads was about equal (dark shaded bars), following the attachment period (light shaded bars); and following the storage and recovery periods (diagonally shaded bars), the number of viable cells loaded to FMB was about twice that attached to Biosilon®. In both types of cell-loaded beads, there was a slight increase in cell number following the storage and recovery periods. The results demonstrate that FMB beads are superior over prior art polystyrene beads for acting as a matrix for high density growth and storage of multipotent cells at RT.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A system for storage and conveyance of viable matrix dependent cells comprising cell-fibrin microbead complexes, culture medium, receptacle for holding said cell-fibrin microbead complexes at a temperature in the range of 16 to 32° C., and a liquid tight closure for sealing said receptacle.

2. The system of claim 1, wherein the fibrin microbeads are cross-linked fibrin microbeads comprising extensively cross-linked fibrin(ogen).

3. The system of claim 1, further comprising serum.

4. The system of claim 1, wherein the receptacle is filled with the culture medium and the cell-fibrin microbead complexes, such that, the volume of the receptacle that is unoccupied by the cell-fibrin microbead complexes is filled with the culture medium.

5. A method of preserving viability of isolated matrix dependent cells, the method comprising the steps of:
(i) providing a preparation of isolated matrix dependent cells;
(ii) culturing the cell preparation of (i) with fibrin microbeads in a culture medium under conditions permitting the cells to bind to the fibrin microbeads, thereby obtaining cell-fibrin microbead complexes; and
(iii) storing the cell-fibrin microbead complexes under sealed conditions at a temperature in the range of 16 to 32° C.

6. The method of claim 5, wherein the cell-fibrin microbead complexes are stored in a receptacle filled with culture medium.

7. The method of claim 6, wherein the culture medium for storing the cells is different from the culture medium in (ii).

8. The method of claim 6, wherein the receptacle further comprises serum.

9. The method of claim 6 wherein the volume of the receptacle that is unoccupied by the cell-fibrin microbead complexes is filled with the culture medium.

10. The method of claim 5, further comprising separating the—fibrin microbead complexes from unbound cells and unbound fibrin microbeads, prior to step (iii).

11. The method of claim 5, wherein the matrix dependent cells are selected from the group consisting of differentiated cells and multipotent progenitor cells.

12. The method of claim 5, wherein the conditions permitting the cells to bind to the fibrin microbeads comprise slow rotary or oscillating incubation at 35 to 37° C., in an environment containing about 21% oxygen and between about 5-10% CO₂.

13. The method of claim 5, wherein the storing is carried out at a ratio of cell-fibrin microbead complexes:culture medium ranging from 1:5 to 1:50 (v/v).

14. The method of claim 5, wherein the storing is carried out for 3 to 21 days, such that, during storage the viability of the of isolated matrix dependent cells is preserved.

15. The method of claim 11, wherein the cells are multipotent progenitor cells and the capacity of the cells to differentiate is maintained.

16. The method of claim 15, further comprising implanting the cell-fibrin microbead complexes in a subject in need thereof.

17. The method of claim 16, wherein the cells are mesenchymal stem cells selected from the group consisting of: autologous mesenchymal stem cells, homologous mesenchymal stem cells, and xenogeneic mesenchymal stem cells.

18. The method of claim 16, wherein the cells are recovered from the cell-fibrin microbead complexes prior to being implanted.

19. The method of claim 16 for treating a disease.

20. The method of claim 19, wherein the disease is selected from the group consisting of: a cartilage or bone defect, spinal cord injury, periodontal disease and myocardial infarction.
